(19) United States
(12) Patent Application Publication
(10) Pub. No.: US 2005/0265971 A1
(43) Pub. Date: Dec. 1, 2005

(54) Agent for treatment of breast cancer and pancreatic cancer comprising herpes simplex virus mutant

(76) Inventors: Akimasa Nakao, Nagoya-shi (JP); Yukihiro Nishiyama, Nagoya-shi (JP)

Correspondence Address: BIRCH STEWART KOLASCH & BIRCH, PO BOX 747, FALLS CHURCH, VA, 22040-0747, US

(21) Appl. No.: 11/076,871

(22) Filed: Mar. 11, 2005

(30) Foreign Application Priority Data
May 26, 2004 (JP) 2004-156475

(51) Int. Cl. A61K 48/00

(52) U.S. Cl. 424/93.2; 424/93.6

(57) ABSTRACT

There is provided a novel therapeutic agent for pancreatic cancer, metastatic breast cancer and recurrent breast cancer in human. The present invention provides a method for treating a patient having pancreatic cancer, metastatic breast cancer or recurrent breast cancer comprising administrating to the patient a herpes simplex virus mutant where UL56 gene Is inactivated. Preferably, the herpes simplex virus mutant is a herpes simplex virus type 1 HF10 strain.

### BACKGROUND OF THE INVENTION

1. Field of the Invention

The present invention relates to treatment of human breast cancer and pancreatic cancer, particularly metastatic breast cancer and recurrent breast cancer using attenuated virus.

2. Description of the Related Art

Breast cancer is one of the most common and feared cancers. Although many methods for treatment such as novel chemotherapeutic agents and hormone preparations have been developed, prognosis of the patients with advanced or metastatic/recurrent breast cancer remains poor. In Japan, the number of breast cancer patient is in an increasing due to the changes of food, life style, stress and so on. Accordingly, there has been a demand for a novel strategy for the treatment of metastatic breast cancer and recurrent breast cancer.

Pancreatic cancer is one of the most lethal of human malignancies, and Is difficult to detect at early stage. Common strategy in the treatment of pancreatic cancer is surgery, radiotherapy and chemotherapy, and combinations thereof. Currently, the most common strategy is treatment with gemcitabine. However, the prognosis of pancreatic cancer is still poor, with the five-year survival rate of 9.5%. Accordingly, a novel strategy for effective treatment of pancreatic cancer is required.

Attenuated herpes simplex virus (HSV) have been proposed for use in the treatment of cancer. This virus has the following useful characteristics: 1) HSV is able to infect to nearly all kinds of cells; 2) infecting ability of HSV is higher than other viruses such as adenovirus and adeno-accompanied virus (AAV); 3) all fundamental sequences of HSV genome have already been determined: 4) HSV is able to kill infected cells at a low dose; and 5) antiviral agent to HSV is available.

Many HSV mutants have been modified according to genetic engineering throughout the world and evaluated for their effect as agents for treatment in the therapy of cancer. Examples of such HSV mutants are G207, NV1020 and MGH1. G207 has deficiency of gamma 34.5 (γ₁ 34.5) of both alleles and insertion of Escherichia coil lacz gene at UL39, while NV1020 has deficiency at one of the gamma 34.5 allele and also has deficiency in an internal repetitive region and deficiency of UL23. All of them have been prepared for attenuation of virus by inactivating the gamma 34.5 allele. However, those viruses are too much attenuated and are unable to sufficiently kill malignant cells and, therefore, nearly all clinical tests using NV1020 and G207 in the United States failed to achieve a sufficient effect in terms of shrinking effect for cancer except safety issues.

It is disclosed that an attenuated mutant of herpes simplex virus (HSV) type 1 in which exogenous suicide gene is incorporated is effective for the treatment of cancer (Japanese Patent Laid-Open No. 2002/218,975). There is another report that an attenuated mutant of herpes simplex virus in which a certain type of gene is inactivated can elongate the survival period of nude mice into which tumor cells are inoculated (Teshigahara, et al., J. Surg. Oncol., 85: 42-47, 2004; WO 02/092826). However, it has not been possible to predict whether herpes simplex virus mutants as such actually have efficacy in the treatment of human cancer and whether they are able to be used for the treatment of metastatic or recurrent cancer.

### SUMMARY OF THE INVENTION

An object of the present invention is to provide a novel agent for the treatment of breast cancer and pancreatic cancer, in particular metastatic breast cancer or recurrent breast cancer.

The present inventors have carried out a phase I clinical test in which an attenuated herpes simplex virus was inoculated to subcutaneous tumor site of recurrent breast cancer for the patients having metastatic breast cancer and found that the virus reduced the metastatic focus without affecting harmful influence to the patients. Thus, in one aspect, the present invention provides a method for treating human metastatic breast cancer or recurrent breast cancer comprising a herpes simplex virus mutant where UL56 gene is inactivated. Preferably, the herpes simplex virus mutant is a herpes simplex virus type 1 HF 10 strain.

### BRIEF DESCRIPTION OF DRAWINGS

FIG. 1 illustrates the structure of HSV HF10 used in the present invention.

FIGS. 2A and 2B show an ultrasonic picture of subcutaneous metastasis of breast cancer of the patient No. 3.

FIGS. 3A, 3B, 3C and 3D show specimens of tumor sites of patients stained with hematoxylin-eosin.

FIG. 4 shows immunostaining with an anti-HSV antibody of a specimen of tumor site to which HSV HF10 was injected.

### DETAILED DESCRIPTION OF PREFERRED EMBODIMENTS

UL56 protein of herpes simplex virus is a membrane protein of a tail-anchored type II localized in Golgi apparatus and endosome. Although function of UL56 protein has not been well clarified yet, it is likely to play a role in the vesicle transportation in virus-infected cells (J. Virol 76: 6718-6728, 2002), suggesting that UL56 may possibly be involved in antegrade axone transportation of virus envelope glycoprotein.

A herpes simplex virus mutant in which UL56 gene is inactivated is able to be prepared either by mutation or deletion of a part of UL56 gene or by insertion of another gene fragment into UL56 gene by way of a known method. An example of such a method is a homologous gene introduction method using a gene fragment in which the sequence to be mutated is flanked by homogeneous sequences. It is also possible to use a naturally generated mutant in which UL56 gene is inactivated.

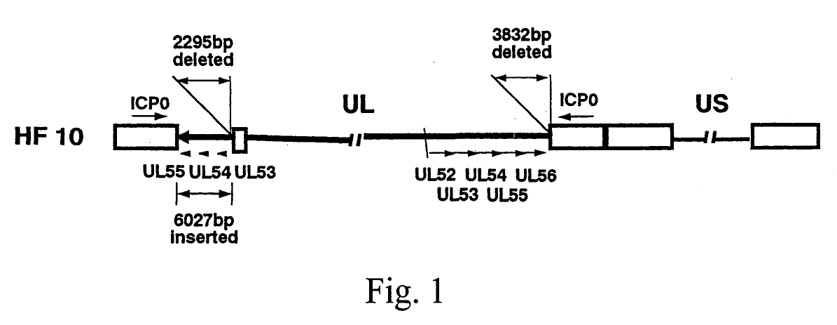

One of the preferred examples of such a mutant is an HF10 strain (see, for example, Arch. Virol., 148 (4); 813-825, 2003). HF10 is derived from an HF strain of HSV-1 and is a clone which causes an extensive cell membrane fusion in infected cells. Genome structure of HF10 is shown in FIG. 1. Its characteristics are as follows: (i) a 3,832-base pair (bp) between nucleotides 116,514 and 120,347 is deleted which indicates that HF10 contains a complete open reading frame (ORF) of ICP0 but has a partial deletion in the UL56 ORF and promoter is completely deleted; and (ii) sequences between 6,025 and 8,319 are deleted from the TRL and the 6,027 bp DNA from nucleotide 110,488 to 116,514 is inserted in an inverted orientation. As a result, the HF10 contains two complete copies of UL53, UL54 and UL55, one complete copy and one partial copy of UL52 and two incomplete copies of UL56 containing no promoter (Arch. Virol., 148(4): 813-825, 2003). HF10 has been reported to elongate the survival period of nude mice in which tumor cells derived from colon cancer, breast cancer, melanoma and sarcoma are inoculated (J. Surg. Oncol., 85: 42-47, 2004). Further, HF10 was nontoxic when intraperitoneally inoculated to mice.

Herpes simplex virus mutant of the present invention in which UL56 gene is inactivated can be grown by a known method. For example, Vero cells derived from renal cells of African green monkey are infected with virus. Round form cells containing virus are collected and subjected to three cycles of freeze-thaw. Cell debris are precipitated by centrifugation to obtain a supernatant containing the virus. The proliferated herpes simplex virus mutant is suspended in sterilized water, sterilized physiological saline solution or any other appropriate buffer to prepare a pharmaceutical preparation of an injectable solution. Further, stabilizers, preservatives or the like may be added to the preparation.

A therapeutic agent comprising herpes simplex virus mutant of the present invention is useful as an agent for the treatment of cancer. The therapeutic agent of the present invention is able to be administered by a direct inoculation to tumor site. The therapeutic agent of the present invention shows a high efficacy particularly as an agent for the treatment of metastatic breast cancer or recurrent breast cancer. As will be shown in the following Examples, cancer cells of the administered site died. Histopathological study showed death of cancer cells to an extent of 30% to 100%.

In addition to breast cancer and pancreatic cancer, the therapeutic agent of the present invention may be used for the treatment of, for example, stomach cancer, esophagus cancer, large bowel cancer, liver cancer, and skin cancer.

The herpes simplex virus mutant used in the therapeutic agent of the present invention is considerably attenuated and its neurotoxicity is significantly reduced, thus it is expected to cause little or no side effect in patients. In the case that some adverse event is observed, it is possible to administer an anti-herpes agent, such as acyclovir and ganciclovir, to the patients for the sake of safety, because attenuated herpes virus has a high sensitivity to such an anti-herpes agent.

### EXAMPLES

The present invention will be illustrated in more detail by way of Examples below, although the present invention is not limited by those Examples.

### Example 1

### Treatment of Breast Cancer

Patients and Methods

All six patients were female, aged 48 to 76 and diagnosed as breast cancer. While all of them had mastectomy, they recurred and had adjuvant chemotherapy and/or endocrine therapy and/or radiation therapy, The recurrent foci were progressive and metastasized to cutaneous or subcutaneous region and that was pathologically proven to be recurrent breast cancer. The profiles of the patients are shown in Table 1.

TABLE 1

| | | | Amount of | Administered | Histological | Histopathological | |
|---|---|---|---|---|---|---|---|
| No. | Age | Sex | Virus (pfu) | Times | Response | Observation | Toxicity |
| 1 | 61 | female | 1 × 10⁴ | 1 | 1b | invasive ductal | — |
| | | | | | | carcinoma | |
| 2 | 62 | female | 1 × 10⁵ | 1 | 1a | invasive ductal | — |
| | | | | | | carcinoma | |
| 3 | 48 | female | 1 × 10⁵ | 3 | 2 | invasive ductal | — |
| | | | | | | carcinoma | |
| 4 | 66 | female | 5 × 10⁵ | 1 | 1b | invasive ductal | — |
| | | | | | | carcinoma | |
| 5 | 72 | female | 5 × 10⁵ | 3 | 2 to 3 | mucinous | — |
| | | | | | | carcinoma | |
| 6 | 76 | female | 5 × 10⁵ | 3 | judgment | scirrhous | — |
| | | | | | impossible | carcinoma | |

In order to evaluate a therapeutic possibility of HF10, which is a herpes simplex virus mutant of the present invention, in human diseases, toxicity and efficacy of HF10 were tested in six patients with subcutaneous recurrent breast cancer as a phase 1 test. For each patient, HF10 diluent in various doses was injected to a test tumor site. For patient No. 1: 1×10⁴ pfu/0.5 ml; for patient No. 2: 1×10⁵ pfu/0.5 ml; for patient No. 3: 1×10⁵ pfu/0.5 ml for three days; for patient No. 4: 5×10⁵ pfu/0.5 ml; for patient Nos. 5 and 6: 5×10⁵ pfu/0.5 ml for three days. As a control, 0.5 ml of sterile physiological saline solution was injected to the second tumor site. All patients were monitored for local and systemic adverse effects, and body temperature, local heat, size of tumor site, reddishness and degree of inflammation were checked.

Assessment of Therapeutic Response

Assessment for the therapeutic response followed the histopathological criteria for therapeutic effect prepared by the Japanese Breast Cancer Society.

Results

All six patients showed good tolerability in the clinical test. Cancer cells were killed to an extent of 30 to 100% and histopathologically denatured. In all patients, local and systemic adverse side effect was not observed.

Macroscopic Findings

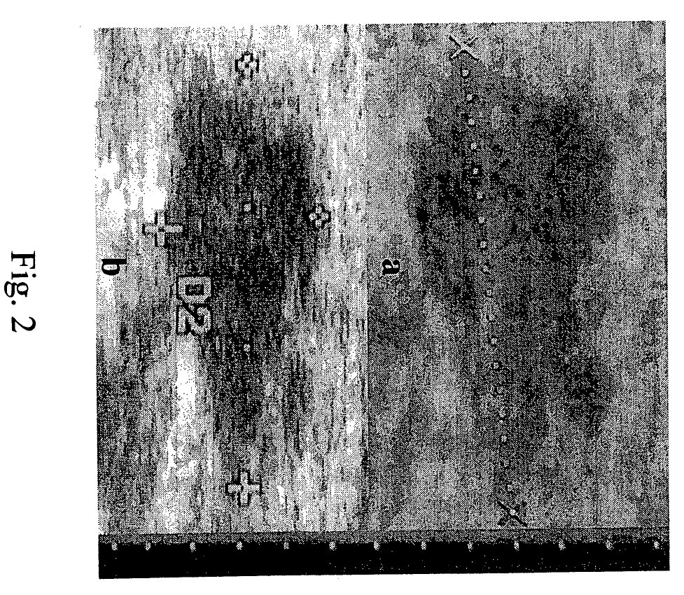

Microscopically, in the recurrent tumor site of the patient No. 3 to whom 1×10⁵ pfu/0.5 ml of HF10 was injected for three days, the height of the tumor site decreased. FIG. 2 shows the image of the ultrasonic test of subcutaneous metastasis of breast cancer in the patient No. 3. FIG. 2A shows the image of ultrasonic test for the same focus before injection of HF10 while FIG. 2B shows the same after 10 days from the injection. As compared with FIG. 2A, the height of the tumor site in FIG. 25 decreased about 30%. In other patients, macroscopic sizes of the tumor site did not show obvious decrease.

Blood Tests

Blood tests of the patients were carried out after 0, 1, 3, 7, 14 and 21 day(s) from injection of the virus to measure white blood cells, HSV IgG, NK, IL10, IL12, IFNα, IFNβ, etc. No change was observed in any of those values.

Histopathological Observation

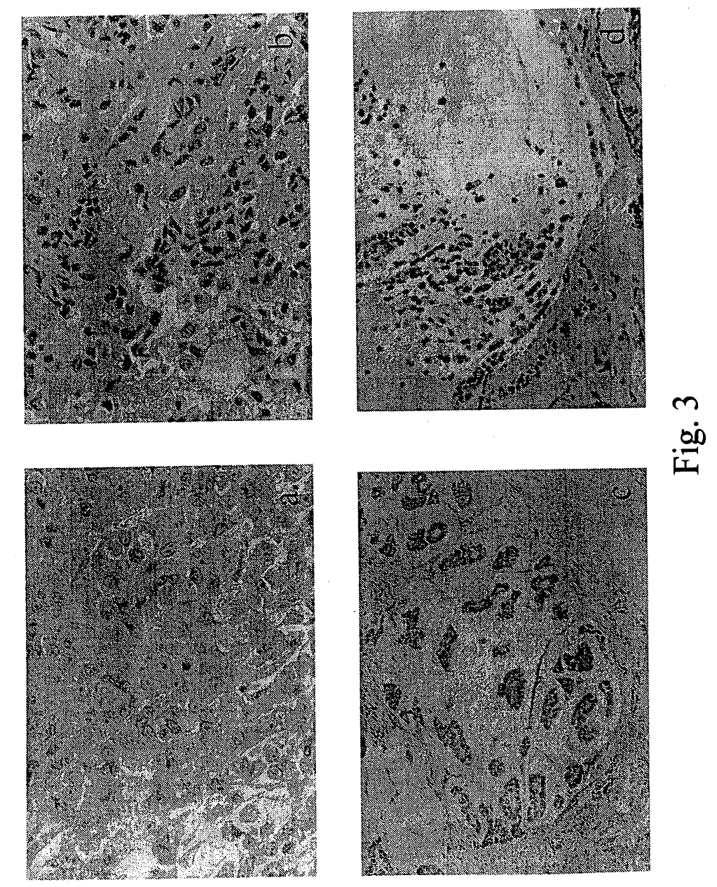

After 14 days from injection of the virus, the tumor site was excised and a histopathologically tested by hematoxylin-eosin (HE) staining. FIG. 3A shows an HE-staining of the specimen before injection of HF10 in the patient No. 3 presenting invasive ductal carcinoma as a result of subcutaneous metastasis of breast cancer. FIG. 3B shows a tissue excised after 14 days from injection of HF10, showing that about two thirds of malignant cells died. Inclusion bodies are noted in breast cancer cells. FIG. 3C shows HE-staining of the specimen before injection of HF10 in the patient No. 5 who has mucinous carcinoma as a result of subcutaneous metastasis of breast cancer. Tumor cells are present around the mucin. FIG. 3D shows that malignant cells completely died after 14 days from injection of HF10.

As a whole, about 30 to 100% of malignant cells died out by injection of herpes simplex virus mutant and, in the patient Nos. 1 to 5, no cell death was noted in the tumor site to which a physiological saline solution was injected. In the patient No. 6, there was nearly no cancer cells in the recurrent tumor site and, therefore, the effect of the virus was not able to be judged.

Immunostaining Method

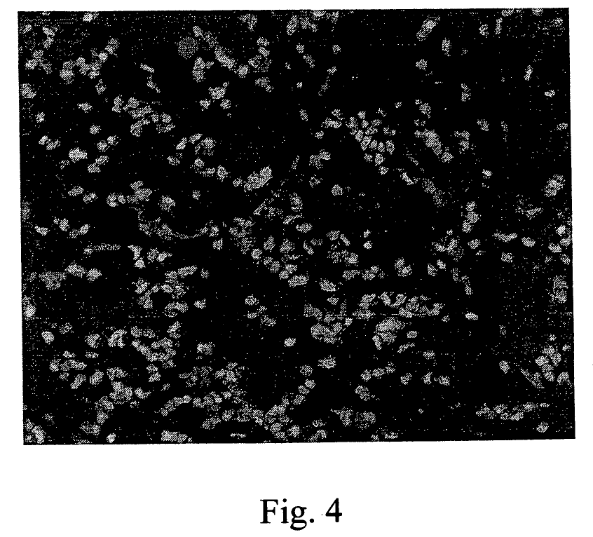

The tumor site to which the virus was injected was excised and immunostained with anti-herpes simplex virus type 1 antibody (DAKO Corporation, Glostrup, Denmark). In all of the patients, presence of viral infection was found to be limited to breast cancer cells (FIG. 4). In addition, in all of the six patients, virus was noted in the breast cancer cells from virus-treated tumor site, while antigen-staining was not observed in the adjacent normal tissues. Since each patient was positive for anti-HSV antibody in blood before injection of the virus, it is noted that the ability of herpes simplex virus mutant HF10 to replicate in tumor cells is not blocked by the previous exposure to HSV.

### Example 2

### Treatment of Pancreatic Cancer

All two patients were male, aged 60 and 68 diagnosed as pancreatic cancer by intra operative histopathological findings. Virus (HF10) injection was performed into tumor foci after the decision of inapplicability for respectable tumor due to peritoneum metastasis, and catheter was inserted into the tumor foci. Consecutive virus injection was performed on POD1 and POD2 using the catheter. The profiles of the patients and result of the test are shown in the table below.

| | | | Amount | | | | |
|---|---|---|---|---|---|---|---|
| | | | of Virus | Administered | Histological | Histopathological | |
| No | Age | Sex | (pfu) | Time | Response | Observation | Toxicity |
| 1 | 68 | Male | 1 × 10⁵ | 3 | Unknown | Adenocarcinoma | — |
| 2 | 60 | Male | 1 × 10⁵ | 3 | Unknown | Adenocarcinoma | — |

Until the time point of 2 months after the first injection, no progression of disease was observed.

This application claims priority to Japanese Patent Application No. 2004-156475, filed on May 26, 2004. This application is hereby incorporated by reference herein in its entirety, including the drawings.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

It will be readily apparent to one skilled in the art that varying substitutions and modification may be made to the invention disclosed herein without departing from the scope and spirit of the invention. Thus, such additional embodiments are within the scope of the present invention and the following claims.

1. A method for treating a patient having pancreatic cancer, metastatic breast cancer or recurrent breast cancer comprising administrating to the patient a herpes simplex virus mutant where UL56 gene is inactivated.

2. The method as claimed in claim 1 wherein the herpes simplex virus mutant is a herpes simplex virus type 1 HF 10 strain.
